(19) United States
(12) Patent Application Publication
(10) Pub. No.: US 2026/0069276 A1
(43) Pub. Date: Mar. 12, 2026

(54) OCCLUSIVE MEDICAL DEVICE

(71) Applicant: BOSTON SCIENTIFIC SCIMED, INC., MAPLE GROVE, MN (US)

(72) Inventors: Neal Eidenschink, Wayzata, MN (US); Christopher J. Clark, St. Michael, MN (US); Ted William Pelzer, Otsego, MN (US)

(73) Assignee: BOSTON SCIENTIFIC SCIMED, INC., MAPLE GROVE, MN (US)

(21) Appl. No.: 19/324,596

(22) Filed: Sep. 10, 2025

Related U.S. Application Data
(60) Provisional application No. 63/693,339, filed on Sep. 11, 2024.

(51) Int. Cl. A61B 17/12

(52) U.S. Cl. CPC A61B 17/12122; A61B 17/12172

(57) ABSTRACT

Example occlusive implants are disclosed. An example occlusive implant includes an expandable framework configured to shift between a collapsed configuration and an expanded configuration, wherein the framework includes a proximal end region and a distal end region. Further, the occlusive implant includes an occlusive member disposed along at least a portion of the expandable framework, wherein a distal extent of the occlusive member extends circumferentially around the framework in a wave configuration.

### CROSS REFERENCE TO RELATED APPLICATIONS

This application claims the benefit of priority of U.S. Provisional Application No. 63/693,339 filed Sep. 11, 2024, the entire disclosure of which is hereby incorporated by reference.

### TECHNICAL FIELD

The disclosure relates generally to medical devices and more particularly to left atrial appendage closure devices.

### BACKGROUND

The left atrial appendage is a small organ attached to the left atrium of the heart as a pouch-like extension. In patients suffering from atrial fibrillation, the left atrial appendage may not properly contract with the left atrium, causing stagnant blood to pool within its interior, which can lead to the undesirable formation of thrombi within the left atrial appendage. Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation are found in the left atrial appendage. As a treatment, medical devices have been developed which are positioned in the left atrial appendage and deployed to close off the ostium of the left atrial appendage. Over time, the exposed surface(s) spanning the ostium of the left atrial appendage becomes covered with tissue (a process called endothelization), effectively removing the left atrial appendage from the circulatory system and reducing or eliminating the number of thrombi which may enter the blood stream from the left atrial appendage. A continuing need exists for improved medical devices and methods to monitor and control thrombus formation within the left atrial appendage of patients suffering from atrial fibrillation.

### SUMMARY

An example occlusive implant includes an expandable framework configured to shift between a collapsed configuration and an expanded configuration, wherein the framework includes a proximal end region and a distal end region. Further, the occlusive implant includes an occlusive member disposed along at least a portion of the expandable framework, wherein a distal extent of the occlusive member extends circumferentially around the framework in a wave configuration.

In addition or alternatively, wherein the distal extent of the occlusive member includes a plurality of peaks and a plurality of valleys, and wherein each of the plurality of peaks extends distal of each of the plurality of valleys.

In addition or alternatively, wherein the plurality of peaks and the plurality of valleys extend circumferentially around an outer surface of the expandable framework in an alternating configuration.

In addition or alternatively, wherein the expandable framework includes a plurality of anchor members disposed along the distal end region of the expandable framework.

In addition or alternatively, wherein the plurality of anchor members includes a first row of anchor members and a second row of anchor members.

In addition or alternatively, wherein the first row of anchor members is positioned proximal of the second row of anchor members.

In addition or alternatively, wherein each of the first row of anchor members and each of the second row of anchor members are coupled to an individual strut of the expandable framework.

In addition or alternatively, wherein each of the first row of anchor members and each of the second row of anchor members extends circumferentially around the longitudinal axis of the expandable member in an alternating configuration.

In addition or alternatively, wherein a portion of the occlusive member adjacent each peak is attached to an individual anchor of the second row of anchor members.

In addition or alternatively, wherein a distal end portion of each of the second row of anchor members extends through the portion of the occlusive member adjacent each peak.

In addition or alternatively, wherein a portion of the occlusive member adjacent each valley is attached to an individual anchor of the first row of anchor members.

In addition or alternatively, wherein a distal end portion of each of the first row of anchor members extends through the portion of the occlusive member adjacent each valley.

Another example occlusive implant for occluding a left atrial appendage includes an expandable framework configured to shift between a collapsed configuration and an expanded configuration, wherein the framework includes a plurality of interconnected struts having a proximal end region and a distal end region. The occlusive implant further includes an occlusive member disposed along at least a portion of the expandable framework, wherein a distal extent of the occlusive member includes a plurality of peaks extending circumferentially around at least a portion of the outer surface of the expandable framework.

In addition or alternatively, wherein the distal extent of the occlusive member includes a plurality of valleys, and wherein the plurality of peaks and the plurality of valleys extend around at least a portion of the outer surface of the expandable framework in an alternating configuration.

In addition or alternatively, wherein each of the plurality of peaks extends distal of each of the plurality of valleys.

In addition or alternatively, wherein the expandable framework includes a first row of anchor members and a second row of anchor members, and wherein each of the first row of anchor members and each of the second row of anchor members extends circumferentially around the longitudinal axis of the expandable framework, and wherein the first row of anchor members is positioned proximal to the second row of anchor members.

In addition or alternatively, wherein each of the first row of anchor members and each of the second row of anchor members extends circumferentially around the longitudinal axis of the expandable member in an alternating configuration.

In addition or alternatively, wherein a portion of the occlusive member adjacent each valley is attached to an individual anchor of the first row of anchor members.

In addition or alternatively, wherein a portion of the occlusive member adjacent each peak is attached to an individual anchor of the second row of anchor members.

An example method for occluding a left atrial appendage includes advancing an occlusive implant to the left atrial appendage. Further, the occlusive implant includes an expandable framework configured to shift between a collapsed configuration and an expanded configuration, wherein the framework includes a proximal end region and a distal end region. Further, the occlusive implant includes an occlusive member disposed along at least a portion of the expandable framework, wherein a distal extent of the occlusive member extends circumferentially around the framework in a wave configuration.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a plan view of an example occlusive implant;

FIG. 2 illustrates an example occlusive implant positioned in the heart;

FIG. 3 illustrates an example occlusive implant positioned in the left atrial appendage;

FIG. 4 illustrates a portion of the occlusive implant shown in FIG. 1;

FIG. 5 illustrates a portion of another example occlusive implant;

FIG. 6 illustrates a portion of another example occlusive implant;

FIG. 7 illustrates a portion of another example occlusive implant;

FIG. 8 illustrates a portion of another example occlusive implant; and

FIG. 9 illustrates a portion of another example occlusive implant.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed disclosure. Those skilled in the art will recognize that the various elements described and/or shown may be arranged in various combinations and configurations without departing from the scope of the disclosure. The detailed description and drawings illustrate example embodiments of the claimed disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The occurrence of thrombi in the left atrial appendage during atrial fibrillation may be due to stagnancy of blood pooling in the left atrial appendage. The pooled blood may still be pulled out of the left atrium by the left ventricle, however less effectively due to the irregular contraction of the left atrium caused by atrial fibrillation. Therefore, instead of an active support of the blood flow by a contracting left atrium and left atrial appendage, filling of the left ventricle may depend primarily or solely on the suction effect created by the left ventricle. However, the contraction of the left atrial appendage may not be in sync with the cycle of the left ventricle. For example, contraction of the left atrial appendage may be out of phase up to 180 degrees with the left ventricle, which may create significant resistance to the desired flow of blood. Further still, most left atrial appendage geometries are complex and highly variable, with large irregular surface areas and a narrow ostium or opening compared to the depth of the left atrial appendage. These aspects as well as others, taken individually or in various combinations, may lead to high flow resistance of blood out of the left atrial appendage.

In an effort to reduce the occurrence of thrombi formation within the left atrial appendage and prevent thrombi from entering the blood stream from within the left atrial appendage, it may be desirable to develop medical devices and/or occlusive implants that seal the left atrial appendage from the heart and/or circulatory system, thereby lowering the risk of stroke due to thrombolytic material entering the blood stream from the left atrial appendage. Further, it may be desirable to configure the left atrial appendage occlusive implant to include an occlusive member which seals the left atrial appendage while also minimizing deployment forces of the occlusive implant from its respective delivery system. Example medical devices and/or occlusive implants configured to seal the left atrial appendage (or other similar openings) while minimizing deployment forces of the occlusive implant from its respective delivery system are disclosed herein.

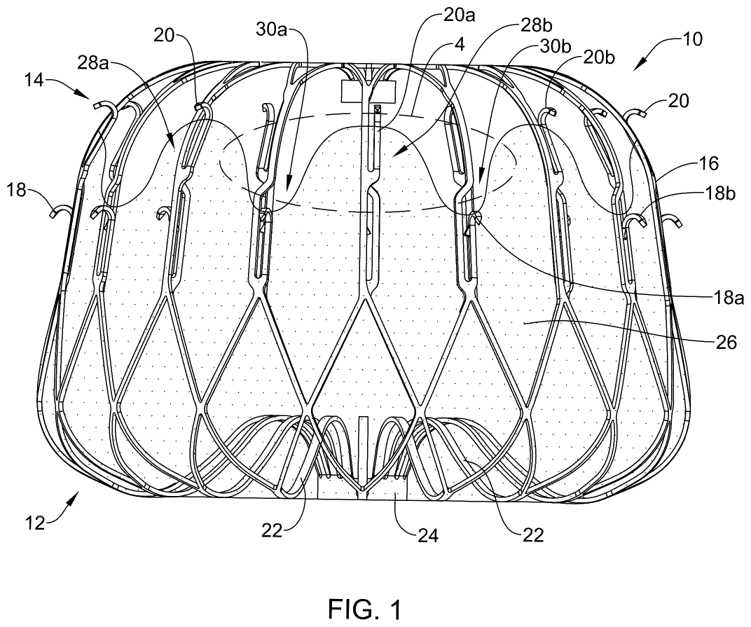

FIG. 1 illustrates an example occlusive implant 10 having a proximal end region 12 and a distal end region 14. The occlusive implant 10 may include an expandable framework 16 formed from a plurality of interconnected strut members extending from the proximal end region 12 to the distal end region 14.

FIG. 1 further illustrates that the expandable framework 16 may include a plurality of anchor members 18, 20 disposed about a periphery of the expandable framework 16. The plurality of anchor members 18, 20 may extend radially outward from the expandable framework 16. In some embodiments, at least some of the plurality of anchor members 18, 20 may each have and/or include a body portion and a tip portion projecting circumferentially therefrom, as shown in FIG. 1. Some suitable, but non-limiting, examples of materials for the expandable framework 16 and/or the plurality of anchor members 18, 20 are discussed below.

FIG. 1 further illustrates that the plurality of anchor members 18, 20 may be arranged into a first row of anchor members 18 and a second row of anchor members 20, whereby the second row of anchor members 20 may be longitudinally offset from the first row of anchor members 18. In the example expandable framework 16 shown in FIG. 1, it can be appreciated that the first row of anchor members 18 may be positioned proximal to the second row of anchor members 20.

Additionally, FIG. 1 illustrates that the distal anchor members 20 and the proximal anchor members 18 may be arranged in a repeating (e.g., alternating) pattern whereby each distal anchor 20 is separated from an adjoining distal anchor 20 by a proximal anchor 18. For example, moving circumferentially around the longitudinal axis of the expandable framework 16, FIG. 1 illustrates a distal anchor 20a, followed by a proximal anchor 18a, followed by a distal anchor 20b, followed by a proximal anchor 18b. It can be appreciated that this alternating pattern (e.g., distal anchor, proximal anchor, distal anchor, proximal anchor, etc.) may be repeated circumferentially around the expandable framework 16. Further, in other examples, the expandable framework 16 may include more than two rows of anchor members 18, 20. For example, in some instances the expandable framework 16 may include 1, 2, 3, 4 or more rows of anchor members.

In some examples, the distal anchor members 20 and the proximal anchor members 18 may be arranged in a variety of repeating (e.g., alternating) patterns. For example, moving circumferentially around the longitudinal axis of the expandable framework 16, a pair of distal anchors 20a may be followed by a proximal anchor 18a, followed by another pair of distal anchors 20a, followed by a proximal anchor 18a, etc. Alternatively, moving circumferentially around the longitudinal axis of the expandable framework 16, a pair of distal anchors 20a may be followed by a pair of proximal anchors 18a, followed by another pair of distal anchors 20a, followed by another pair of proximal anchors 18a, etc. Alternatively, moving circumferentially around the longitudinal axis of the expandable framework 16, a distal anchor 20a may be followed by a pair of proximal anchors 18a, followed by a distal anchor 20a, followed by another pair of proximal anchors 18a, etc.

In some examples, the expandable framework 16, the plurality of anchor members 18, 20 and/or other members/components of the expandable framework 16 disclosed herein may be integrally formed and/or cut from a unitary (e.g., monolithic) member. In some embodiments, the expandable framework 16 and the plurality of anchor members 18, 20 may be integrally formed and/or cut from a unitary tubular member and subsequently formed and/or heat set to a desired shape in the expanded configuration. In some embodiments, the expandable framework 16 and the plurality of anchor members 18, 20 may be integrally formed and/or cut from a unitary flat member, and then rolled or formed into a tubular structure and subsequently formed and/or heat set to the desired shape in the expanded configuration. Some exemplary means and/or methods of making and/or forming the expandable framework 16 include laser cutting, machining, punching, stamping, electro discharge machining (EDM), chemical dissolution, etc. Other means and/or methods are also contemplated.

As illustrated in FIG. 1, the framework 16 may include one or more struts 22 which are fixedly attached (e.g., welding, glued, soldered, crimped, etc.) to a cylindrical collar 24 (e.g., cylindrical band, cylindrical ring). In some examples, the collar 24 may include a cylindrical metallic ring (e.g., a metallic collar). The collar 24 may include an aperture, the center of which may be generally aligned with the longitudinal axis of the expandable framework 16.

The collar 24 may be constructed of a variety of materials. For example, the collar 24 may be formed from a metal, metal alloy, a polymer, a ceramic or any combinations thereof. Further, in some instances, the collar 24 may be constructed from the same material as the framework 16. For example, the framework 16 (including the struts 22) and the collar 24 may be a monolithic structure whereby the framework 16, struts 22 and the collar 24 may be formed from the same monolithic base material. However, it can be appreciated that, in some examples, the framework 16 and the collar 24 may constructed as separate components, whereby the struts 22 of the framework 16 may be affixed to the collar 24. FIG. 1 illustrates that the struts 22 may be affixed circumferentially around the outer surface of the collar 24. In other words, the struts 22 may be attached to the band at evenly (or unevenly) spaced intervals extending 360 degrees around the circumference of the collar 24.

FIG. 1 further illustrates that the occlusive implant 10 may also include an occlusive member 26 disposed on, disposed over, disposed about, or covering at least a portion of the expandable framework 16. In some embodiments, the occlusive member 26 may be disposed on, disposed over, disposed about or cover at least a portion of an outer (or outwardly-facing) surface of the expandable framework 16. FIG. 1 further illustrates that the occlusive member 26 may extend only partially along the longitudinal extent (e.g., longitudinal axis) of the expandable framework 16.

In some embodiments, the occlusive member 26 may be permeable or impermeable to blood and/or other fluids, such as water. In some embodiments, the occlusive member 26 may include a woven, braided and/or knitted material, a fiber, a sheet-like material, a fabric, a polymeric membrane, a metallic or polymeric mesh, a porous filter-like material, or other suitable construction. In some embodiments, the occlusive member 26 may prevent thrombi (i.e. blood clots, etc.) from passing through the occlusive member 26 and out of the left atrial appendage into the blood stream. In some embodiments, the occlusive member 26 may promote endothelialization after implantation, thereby effectively removing the left atrial appendage from the patient's circulatory system. Some suitable, but non-limiting, examples of materials for the occlusive member 26 are discussed below.

It can be appreciated that, in some examples, the collar 24 may include one or more fixation components configured to allow the occlusive member 26 to attach thereto. For example, the collar 24 may include hooks, holes, tines, etc. configured to allow the occlusive member 26 to attach thereto. As discussed above, in some examples, the occlusive member 26 may be constructed from a fabric, and therefore, may be able to attach onto one or more of the hooks, holes, tines, etc. located on the collar 24.

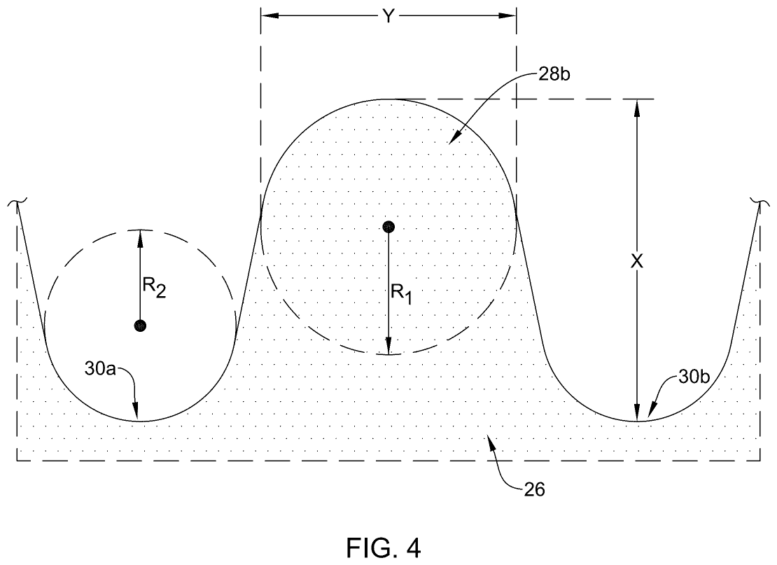

FIG. 1 further illustrates that the distalmost extent (e.g., the distalmost edge) of the occlusive member 26 may include a wave configuration. As will be described in greater detail with respect to FIG. 4, the wave configuration of the distalmost extent of the occlusive member 26 may include alternating pattern of curved peaks and valleys. For example, FIG. 1 illustrates the distalmost extent of the occlusive member 26 including a first curved peak 28a, a first curved valley 30a, a second curved peak 28b and a second curved valley 30b. It can be appreciated that this alternating pattern (e.g., peak, valley, peak, valley, etc.) may be repeated around the circumference of the expandable framework 16.

As disclosed herein, it can be appreciated that the wave configuration of the distalmost extent of the occlusive member 26 may include configurations in which the distal edge of the occlusive member 26 is undulating, curvy, sinusoidal, or includes any type of wave-shaped peaks and wave-shaped valleys. However, in other examples, the peaks and the valleys of the distalmost extent of the occlusive member 26 may also include additional shapes. For example, the distalmost extent (e.g., distalmost edge) of the occlusive member 26 may include peaks and valleys whose shape may be triangular, square, rectangular, ovular, trapezoidal, elliptical, semi-circular, kite-shaped, star-shaped, any variation thereof or combination thereof. Furthermore, in some examples, the distalmost extent of the occlusive member 26 may include non-repeating configurations of any shape variation, including those disclosed herein.

It can be appreciated from FIG. 1 that the valleys and peaks defining the distalmost extent of the occlusive member 26 may be attached to the expandable framework 16 via the anchor members 18, 20. For example, FIG. 1 illustrates that the distalmost extent of the valley 30b may be attached to the expandable framework 16 via the proximal anchor 18a. It can be appreciated that, in some examples, each proximal anchor 18 (e.g., proximal anchor 18a) of the expandable framework 16 may be pushed through the occlusive member 26 at the location of a valley, thereby coupling the occlusive member 26 to the expandable framework 16 and preventing the occlusive member 26 from shifting longitudinally along the outer surface of the expandable framework 16. It can be further appreciated that each valley of the occlusive member 26 may be attached to a proximal anchor 18 around the circumference of the expandable framework 16. Similarly, FIG. 1 illustrates that the distalmost extent of the peak 28b may be attached to the expandable framework 16 via the distal anchor 20a. It can be appreciated that, in some examples, each distal anchor 20 (e.g., distal anchor 20a) of the expandable framework 16 may be pushed through the occlusive member 26 at the location of a peak, thereby coupling the occlusive member 26 to the expandable framework 16 and preventing the occlusive member 26 from shifting longitudinally along the outer surface of the expandable framework 16. It can be further appreciated that each peak of the occlusive member 26 may be attached to a distal anchor 20 around the circumference of the expandable framework 16.

It can be appreciated that attaching the occlusive member 26 to the expandable framework 16 along both a proximal row of anchors 18 and a distal row of anchors 20 may provide a secure attachment of the occlusive member 26 to the expandable framework in addition to providing uniform attachment forces (e.g., uniform tension) to be applied to the occlusive member 26 when positioned along the outer surface of the expandable framework 16. Additionally, it can be appreciated that the peak regions (e.g., peaks 28a, 28b, etc.) which extend distally along the outer surface of the expandable member 16 may improve the sealing capabilities of the occlusive member 10 when positioned in the left atrial appendage. Further, it can be appreciated that not extending the entire distal extent of the occlusive member 26 to the distal length defined by the peak regions (e.g., peaks 28a, 28b, etc.) may reduce deployment forces when the occlusive member 10 is deployed from a deployment device. In other words, the absence of material which define the valley regions (30a, 30b, etc.) of the occlusive member 26 may reduce deployment forces (e.g., frictional forces) of the occlusive member 10 when being deployed from a deployment device.

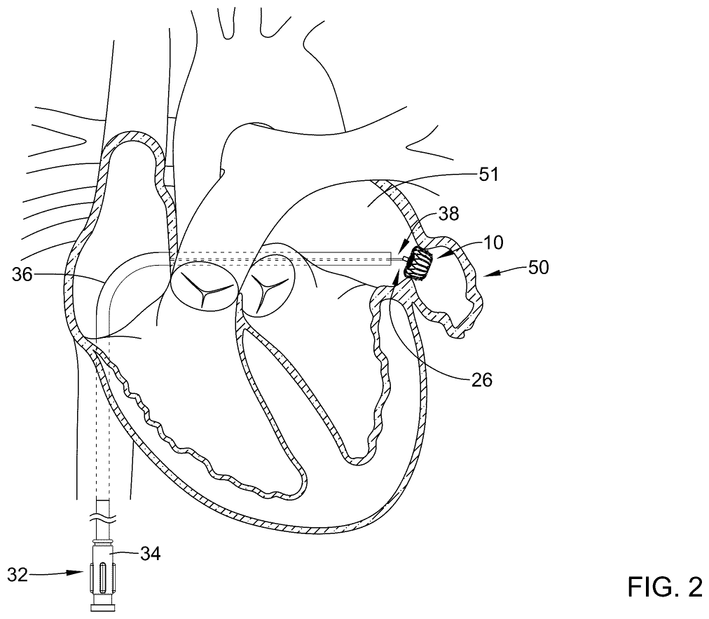

FIG. 2 illustrates that the occlusive implant 10 may be inserted and advanced through a body lumen via an occlusive implant delivery system 32. FIG. 2 further illustrates the occlusive implant 10 being delivered and positioned within the left atrial appendage 50. In some instances, an occlusive implant delivery system 32 may include a delivery catheter 36 which is guided toward the left atrium 51 via various chambers and lumens of the heart (e.g., the inferior vena cava, the right atrium, etc.) to a position adjacent the left atrial appendage 50.

The delivery system 32 may include a hub member 34 coupled to a proximal region of the delivery catheter 36. The hub member 34 may be manipulated by a clinician to direct the distal end region of the delivery catheter 36 to a position adjacent the left atrial appendage 50. In some embodiments, an occlusive implant delivery system 32 may include a core wire 38. Further, a proximal end (e.g., collar 24) of the expandable framework 16 may be configured to releasably attach, join, couple, engage, or otherwise connect to the distal end of the core wire 38. In some embodiments, an end region of the expandable framework 16 (e.g., collar 24) may include a threaded insert coupled thereto. In some embodiments, the threaded insert may be configured to and/or adapted to couple with, join to, mate with, or otherwise engage a threaded member disposed at the distal end of a core wire 38. Other means of releasably coupling and/or engaging the proximal end of the expandable framework 16 to the distal end of the core wire 38 are also contemplated.

It can be appreciated that, in some instances, the core wire 38 (or any other component of the delivery system 32) may be releasably attached to the collar 24. For example, in some instances, the core wire 38 may be releasably attached, engaged, joined, coupled, or otherwise connected to the collar 24, the expandable framework 16, or any other component of the medical device 10 via a variety of different connection methods. For example, it is contemplated that the collar 24 may include a threaded component configured to and/or adapted to couple with, join to, mate with, or otherwise engage a threaded member disposed at the distal end of a core wire 38.

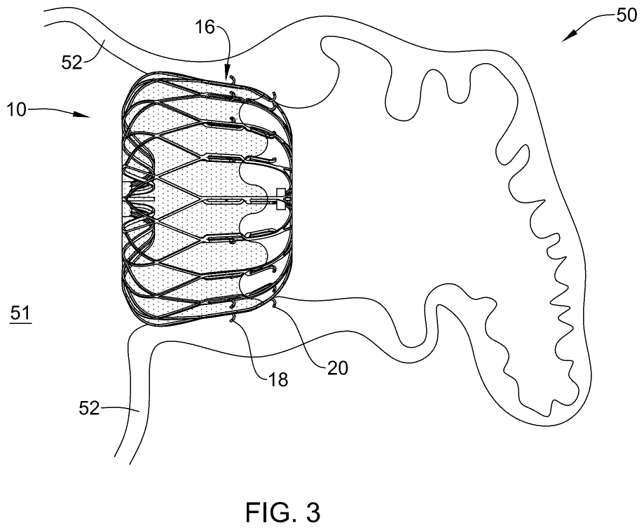

FIG. 3 illustrates the occlusive implant 10 positioned within the left atrial appendage 50 via the delivery catheter 32 (described above with respect to FIG. 2). As discussed above, in some examples, the implant 10 may be configured to shift between a collapsed configuration and an expanded configuration. For example, in some instances, the occlusive implant 10 may be in a collapsed configuration during delivery via an occlusion implant delivery system, whereby the occlusive implant 10 expands to an expanded configuration once deployed from the occlusion implant delivery system.

Additionally, FIG. 3 illustrates that the expandable framework 16 may be compliant and, therefore, substantially conform to and/or be in sealing engagement with the shape and/or geometry of a lateral wall of a left atrial appendage 50 in the expanded configuration. In some embodiments, the occlusive implant 10 may expand to a size, extent, or shape less than or different from a maximum unconstrained extent, as determined by the surrounding tissue 52 and/or lateral wall of the left atrial appendage 50. Additionally, FIG. 3 illustrates that the expandable framework 16 may be held fixed adjacent to the left atrial appendage 50 by one or more anchoring members 18, 20 described herein.

Further, it can be appreciated that the elements of the expandable framework 16 may be tailored to increase the flexibility and compliance of the expandable framework 16 and/or the occlusive implant 10, thereby permitting the expandable framework 16 and/or the occlusive implant 10 to conform to the tissue around it, rather than forcing the tissue to conform to the expandable framework 16 and/or the occlusive implant 10. Additionally, in some instances, it may be desirable to configure the occlusive implant 10 discussed above to include various features, components and/or configurations which improve the sealing capabilities of the occlusive implant 10 within the left atrial appendage 50. For example, as described with respect to FIG. 1 and FIG. 3, configuring the occlusive member 26 with both a proximal row of anchors 18 and a distal row of anchors 20 may provide increased securement of the occlusive member 26 to the expandable framework 16 while also providing increased securement of the occlusive member 26 to the surrounding tissue 52 and/or lateral wall of the left atrial appendage 50. Additionally, it can be appreciated that the peak regions (e.g., peak 28a, 28b, etc.) which extend distally along the outer surface of the expandable member 16 may improve the sealing capabilities of the occlusive member 10 when positioned in the left atrial appendage 50.

FIG. 4 illustrates a detailed view of a portion of the occlusive member 26 of FIG. 1. FIG. 4 illustrates the valley 30a, the peak 28b and the valley 30b which form the wave configuration of the distalmost extent of the occlusive member 26 of FIG. 1. FIG. 4 further illustrates that the peak 28b may include a height “X” (as measured from the valley 30b to the apex of the peak 28b) of about 0.127 millimeters (mm) (0.005 inches) to about 8.89 mm (0.35 inches), or about 0.254 mm (0.010 inches) to about 6.35 mm (0.25 inches), or about 0.381 mm (0.015 inches) to about 5.08 mm (0.20 inches). FIG. 4 illustrates that the peak 28b may include a width “Y” of about 0.254 mm (0.010 inches) to about 12.7 mm (0.50 inches), or about 0.381 mm (0.015 inches) to about 11.43 mm (0.45 inches), or about 1.016 mm (0.04 inches) to about 10.16 mm (0.40 inches), or about 2.032 mm (0.08 inches) to about 7.62 mm (0.30 inches), or about 2.54 mm (0.10 inches) to about 6.35 mm (0.25 inches), or about 3.81 mm (0.15 inches) to about 5.08 mm (0.20 inches), or about 10.567 mm (0.416 inches). Additionally, FIG. 4 illustrates that the peak 28b may have a radius of curvature “R₁” of about 0.127 mm (0.005 inches) to about 5.08 mm (0.200 inches). FIG. 4 further illustrates that each of the valleys 30a, 30b may include a radius of curvature “R₂” of about 0.127 mm (0.005 inches) to about 5.08 mm (0.200 inches). It can be appreciated that each of the peaks and valleys of the wave configuration of the distalmost extent of the occlusive member 26 of FIG. 1 may be within the approximate value ranges of the peak 28b and the valleys 30a, 30b illustrated in FIG. 4.

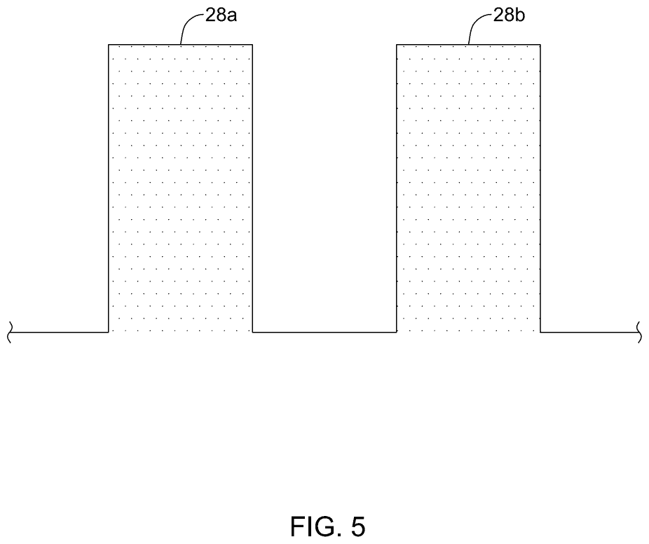

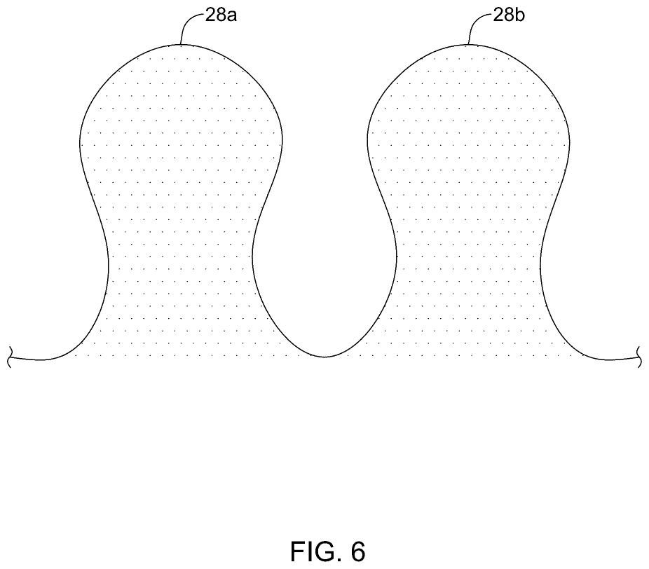

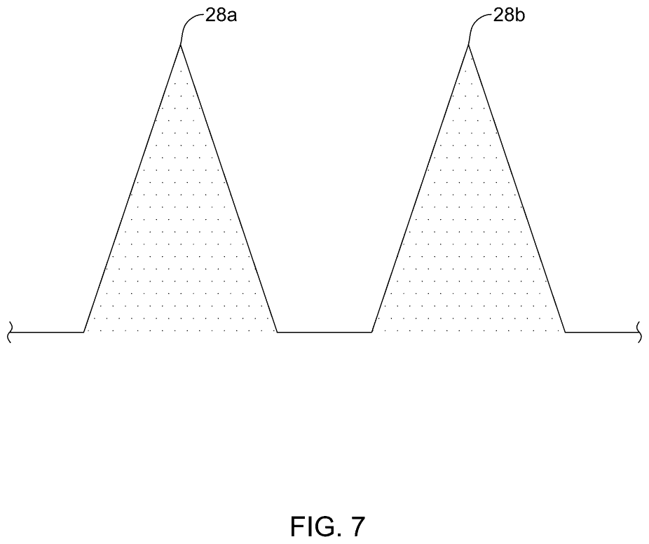

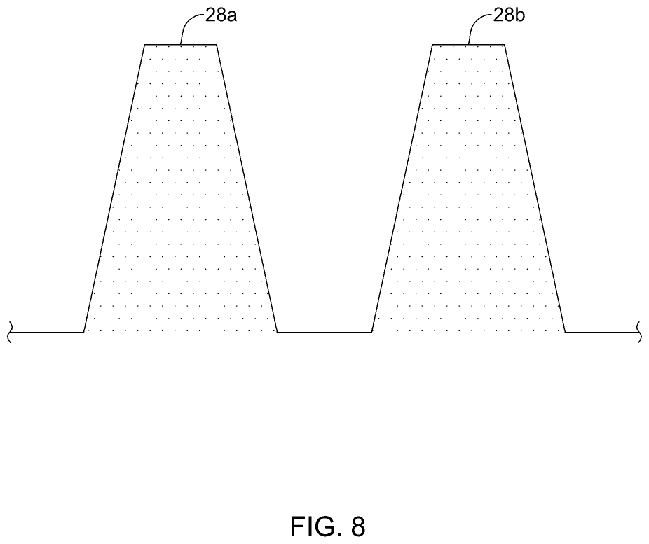

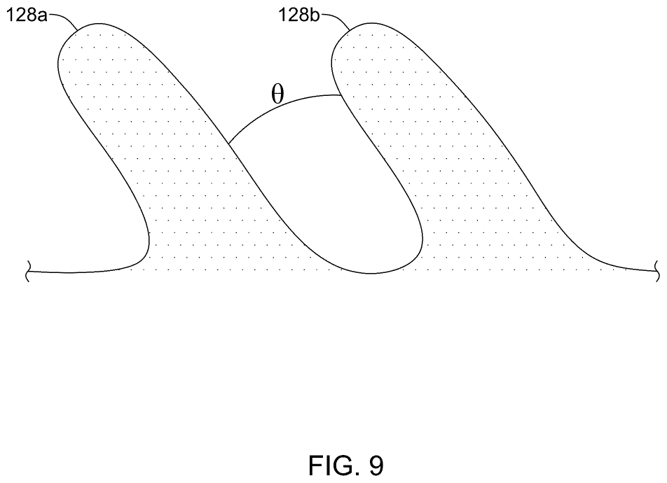

FIGS. 5-9 schematically depict various example arrangements of the distalmost extent of the occlusive member 26 of the occlusive member 10. These examples are in addition to the example arrangement (e.g., wave configuration) of the distalmost extent of the occlusive member 26 described with respect to FIG. 1. The example arrangements of the distalmost extent of the occlusive member 26 shown in FIGS. 5-9 are schematic representations that depict alternative arrangements. The edges/boundaries shown in FIGS. 5-9 are intended to mimic the edge/boundaries of the pattern/arrangement shown in FIG. 1, or a variation thereof, and can be utilized in any of the devices disclosed as alternatives of the distalmost extent of the occlusive member 26. FIG. 9 illustrates a first peak 128a and a second peak 128b. It can be appreciated that the first peak 128a maybe offset from the second peak 128b by about 10 degrees to about 90 degrees, or about 20 degrees to about 80 degrees, or about 10 degrees to about 90 degrees, or about 30 degrees to about 70 degrees, or about 40 degrees to about 60 degrees.

The materials that can be used for the various components of the occlusive implant 10 (and variations, systems or components thereof disclosed herein) and the various elements thereof disclosed herein may include those commonly associated with medical devices. For simplicity purposes, the following discussion refers to the occlusive implant 10 (and variations, systems or components disclosed herein). However, this is not intended to limit the devices and methods described herein, as the discussion may be applied to other elements, members, components, or devices disclosed herein.

In some embodiments, the occlusive implant 10 (and variations, systems or components thereof disclosed herein) may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the occlusive implant 10 (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the occlusive implant 10 (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the occlusive implant 10 (and variations, systems or components thereof disclosed herein) to achieve the same result.

In some embodiments, a degree of Magnetic Resonance Imaging (MRI) compatibility is imparted into the occlusive implant 10 (and variations, systems or components thereof disclosed herein). For example, the occlusive implant 10 (and variations, systems or components thereof disclosed herein) and/or components or portions thereof, may be made of a material that does not substantially distort the image and create substantial artifacts (e.g., gaps in the image). Certain ferromagnetic materials, for example, may not be suitable because they may create artifacts in an MRI image. The occlusive implant 10 (and variations, systems or components disclosed herein) or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the occlusive implant 10 (and variations, systems or components thereof disclosed herein) and/or portions thereof, may be made from or include a polymer or other suitable material. Some examples of suitable polymers may include copolymers, polyisobutylene-polyurethane, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the occlusive implant 10 (and variations, systems or components thereof disclosed herein) may include a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun-types. The type and denier of the yarn chosen may be selected in a manner which forms a biocompatible and implantable prosthesis and, more particularly, a vascular structure having desirable properties.

In some embodiments, the occlusive implant 10 (and variations, systems or components thereof disclosed herein) may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

While the discussion above is generally directed toward an occlusive implant for use in the left atrial appendage of the heart, the aforementioned features may also be useful in other types of medical implants where a fabric or membrane is attached to a frame or support structure including, but not limited to, implants for the treatment of aneurysms (e.g., abdominal aortic aneurysms, thoracic aortic aneurysms, etc.), replacement valve implants (e.g., replacement heart valve implants, replacement aortic valve implants, replacement mitral valve implants, replacement vascular valve implants, etc.), and/or other types of occlusive devices (e.g., atrial septal occluders, cerebral aneurysm occluders, peripheral artery occluders, etc.). Other useful applications of the disclosed features are also contemplated.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An occlusive implant, comprising:
an expandable framework configured to shift between a collapsed configuration and an expanded configuration, wherein the framework includes a proximal end region and a distal end region;
an occlusive member disposed along at least a portion of the expandable framework, wherein a distal extent of the occlusive member extends circumferentially around the framework in a wave configuration.

2. The occlusive implant of claim 1, wherein the distal extent of the occlusive member includes a plurality of peaks and a plurality of valleys, and wherein each of the plurality of peaks extends distal of each of the plurality of valleys.

3. The occlusive implant of claim 2, wherein the plurality of peaks and the plurality of valleys extend circumferentially around an outer surface of the expandable framework in an alternating configuration.

4. The occlusive implant of claim 2, wherein the expandable framework includes a plurality of anchor members disposed along the distal end region of the expandable framework.

5. The occlusive member of claim 4, wherein the plurality of anchor members includes a first row of anchor members and a second row of anchor members.

6. The occlusive member of claim 5, wherein the first row of anchor members is positioned proximal of the second row of anchor members.

7. The occlusive member of claim 6, wherein each of the first row of anchor members and each of the second row of anchor members are coupled to an individual strut of the expandable framework.

8. The occlusive implant of claim 7, wherein each of the first row of anchor members and each of the second row of anchor members extends circumferentially around the longitudinal axis of the expandable member in an alternating configuration.

9. The occlusive implant of claim 8, wherein a portion of the occlusive member adjacent each peak is attached to an individual anchor of the second row of anchor members.

10. The occlusive implant of claim 9, wherein a distal end portion of each of the second row of anchor members extends through the portion of the occlusive member adjacent each peak.

11. The occlusive implant of claim 10, wherein a portion of the occlusive member adjacent each valley is attached to an individual anchor of the first row of anchor members.

12. The occlusive implant of claim 11, wherein a distal end portion of each of the first row of anchor members extends through the portion of the occlusive member adjacent each valley.

13. An occlusive implant for occluding a left atrial appendage, comprising:
an expandable framework configured to shift between a collapsed configuration and an expanded configuration, wherein the framework includes a plurality of interconnected struts having a proximal end region and a distal end region;
an occlusive member disposed along at least a portion of the expandable framework, wherein a distal extent of the occlusive member includes a plurality of peaks extending circumferentially around at least a portion of the outer surface of the expandable framework.

14. The occlusive implant of claim 13, wherein the distal extent of the occlusive member includes a plurality of valleys, and wherein the plurality of peaks and the plurality of valleys extend around at least a portion of the outer surface of the expandable framework in an alternating configuration.

15. The occlusive implant of claim 14, wherein each of the plurality of peaks extends distal of each of the plurality of valleys.

16. The occlusive member of claim 15, wherein the expandable framework includes a first row of anchor members and a second row of anchor members, and wherein each of the first row of anchor members and each of the second row of anchor members extends circumferentially around the longitudinal axis of the expandable framework, and wherein the first row of anchor members is positioned proximal to the second row of anchor members.

17. The occlusive implant of claim 16, wherein each of the first row of anchor members and each of the second row of anchor members extends circumferentially around the longitudinal axis of the expandable member in an alternating configuration.

18. The occlusive implant of claim 17, wherein a portion of the occlusive member adjacent each valley is attached to an individual anchor of the first row of anchor members.

19. The occlusive implant of claim 18, wherein a portion of the occlusive member adjacent each peak is attached to an individual anchor of the second row of anchor members.

20. A method for occluding a left atrial appendage, the method comprising:
advancing an occlusive implant to the left atrial appendage, the occlusive implant including:
an expandable framework configured to shift between a collapsed configuration and an expanded configuration, wherein the framework includes a proximal end region and a distal end region;
an occlusive member disposed along at least a portion of the expandable framework, wherein a distal extent of the occlusive member extends circumferentially around the framework in a wave configuration.
